(19) United States
(12) Patent Application Publication
(10) Pub. No.: US 2024/0298948 A1
(43) Pub. Date: Sep. 12, 2024

(54) MUSCLE ACTIVATION AND MOVEMENT DETECTION AND ALERT DEVICE

(71) Applicant: Core Plus Device, LLC, Holland, MI (US)

(72) Inventors: David D. Weller, Holland, MI (US); Kathleen M. Weller, Holland, MI (US); Maureen A. Hogan, Holland, MI (US); Kristin J. Zietlow, West Olive, MI (US)

(21) Appl. No.: 18/592,386

(22) Filed: Feb. 29, 2024

Related U.S. Application Data
(60) Provisional application No. 63/450,553, filed on Mar. 7, 2023.

(51) Int. Cl. A61B 5/22; A61B 5/00; A61B 5/11

(52) U.S. Cl. CPC A61B 5/224; A61B 5/1116; A61B 5/6823; A61B 5/6891; A61B 5/7465; A61B 2560/0223; A61B 2560/0406

(57) ABSTRACT

A muscle activation and movement detection, monitoring, tracking, and alert device includes pivot arms formed into a cross-shape. The cross-shape is concave where a plurality of sensors is configured under the ends of the pivot arms for detecting forces applied to the device from the user's body. The sensors provide instantaneous feedback to the user of strength, angular positioning, duration, exercise tracking, and alerts relating to muscle activation and movement.

This collective feedback delivers a comprehensive system to aide users in improving their strength. This device can detect when the user's muscles are engaged or bodily movement achieved, through measuring orientation, movement, endurance and force exerted by the user's body.

### FIELD OF THE INVENTION

The present invention relates generally to detecting movement of a user's activated muscles during physical strength exercises and more specifically to a muscle activation detection device that alerts the user through feedback via audio, vibratory, lighting, mobile device or computer when a user's muscle movement, stretch, posture and/or strength exercise motion is detected.

### BACKGROUND

There are many physical exercise techniques used to improve strength in the body. A person can improve their physical health through physical therapy or personal strength exercises in many areas including but not limited to neck, pelvic floor, abdominal, cervical scapular and quads. When proper exercises are done in areas like the lower core, it often improves or prevents lower back pain. A person's “core” is the central part of their body. It includes the pelvis, lower back, hips and stomach muscles. These stomach muscles are sometimes are referred to as the abs.

Physical exercises train the muscles in your body to work in harmony. This leads to better balance and steadiness, also called stability. A weak core can not only lead to lower back pain but also cause issues with posture and overworked muscles in other areas of the body. Nerve issues, damage to the intervertebral discs, compression of nerve roots, pulled tendons, and overworked muscles are all lower back issues that can occur due to a weak core and poor posture that can cause pain. This philosophy can apply to many other areas of improving health through physical strength exercises including but not limited to neck, pelvic floor, abdominal, cervical scapular and quads.

Although there are devices in the prior art for detecting body movements, strength and angular positions, like posture, these devices only detect position through either positional orientation only (e.g. internal gyroscope) or a singular point of measurement on the body. They also do not create, count and track progress for a user's exercise routines and overall core strength improvements. They also do not adequately capture relative movement in the body-for instance, how the left side of the back is moving compared to the right side of the back.

Finally, currently available devices often require a person to wear uncomfortable objects or endure painful electromyography (EMG) measurements. Thus, new technology is required to detect high-accuracy muscle strength, endurance, stretches, and exercises, track the routines and repetitions of movements, positions, stretches, and exercises, and finally notify persons when their activation forces and/or physical orientation are not correct for specific movement, strength, endurance, position, stretching and exercise profiles. The combination of these features should offer unique solutions for diagnostic, therapeutic, and preventative physical exercise and therapy techniques.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1A is a perspective view illustrating the muscle activation and movement detection device according to an embodiment of the invention.

FIG. 1B is an exploded view of the muscle activation and movement detection device shown in FIG. 1.

FIG. 1C is a side view of the muscle activation and movement detection device shown in FIG. 1.

FIG. 1D is a cross-sectional view of the muscle activation and movement detection device shown through lines 1D-1D shown in FIG. 1C.

FIG. 2A and FIG. 2B are isometric views showing sensor movement with forces applied.

FIG. 3A, FIG. 3B and FIG. 3C are top, side and isometric views respectively of a muscle activation and movement detection device according to a first alternative embodiment of the invention.

FIG. 4A, FIG. 4B are FIG. 4C are top, side and isometric view respectively of a muscle activation and movement detection device according to a second alternative embodiment of the invention.

FIG. 5A, FIG. 5B, FIG. 5C and 5D are top, exploded, cross-sectional and magnified views respectively of a muscle activation and movement detection device according to a third alternative embodiment of the invention.

FIG. 6 is a perspective view of an alternative embodiment of the invention illustrating two flex sensors across the center of the core activation detection device.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a muscle activation and movement detection, exercise routines, strength tracking, and alert device. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

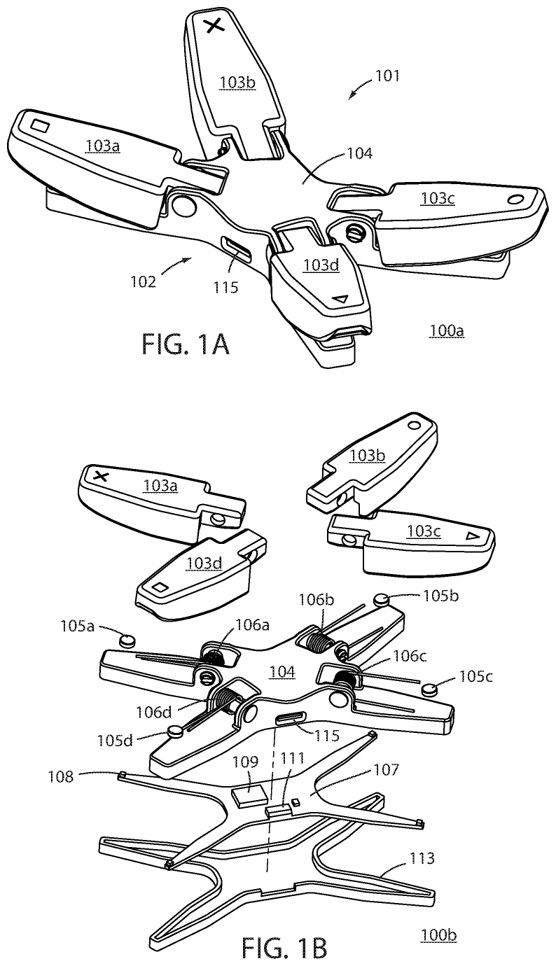

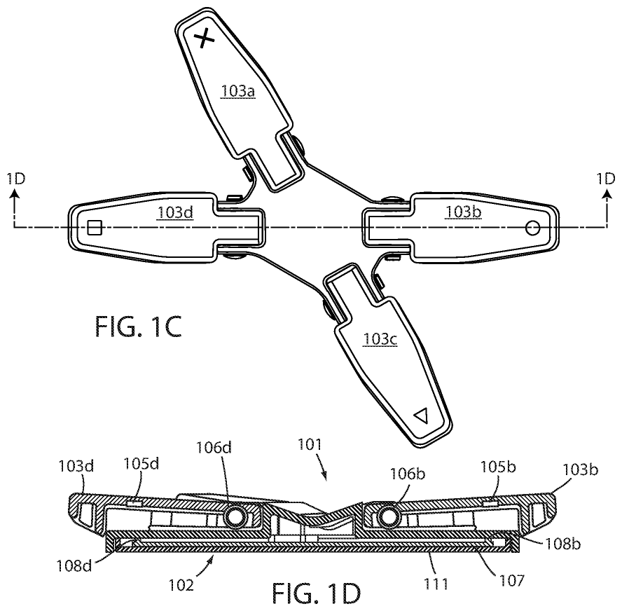

FIG. 1A is a perspective view illustrating a muscle activation and movement detection device according to an embodiment of the present invention. FIG. 1B is an exploded view of the muscle activation and movement detection device shown in FIG. 1. FIG. 1C is a side view of the muscle activation and movement detection device shown in FIG. 1A. FIG. 1D is a cross-sectional view of the muscle activation and movement detection device shown through lines 1D-1D shown in FIG. 1C.

With regard to each of FIG. 1A to FIG. 1D, the angular detection and alert device 100a, 100b, 100c, 100d includes a front side 101 and back side 102. In use, the front side 101 is positioned against the user's body while the back side 102 can remain open or in alternative embodiments can rest within or against a supporting surface such as a chair seat back or within a medical device such as an orthopedic cast or shell. A plurality of semi-flexible or rigid battens such as pivot arms 103a, 103b, 103c, 103d each connect and extend from a central body or housing 104. The pivot arms 103a, 103b, 103c, 103d each extend outwardly and are configured to form a crossed or x-like shape with the housing 104. Housing 104 is concave in shape forming a pocket enabling it to cover the spine area when worn. As best seen in FIG. 1B, the pivot arms 103a, 103b, 103c, 103d bend inwardly so to allow muscle activation and movement detection devices 105a105b, 105c, 105d to form the concave shape when facing toward the front side 101.

Each of the pivot arms 103a, 103b, 103c, 103d include a respective recessed area that is used to house a respective magnet 105a, 105b, 105c, 105d as well as a torsion spring 106a, 106b, 106c, 106d. The torsion spring 106a, 106b, 106c, 106d each allowing its respective pivot arm 103a, 103b, 103c, 103d to move and pivot from its pivot end near the center of the body 104. Each magnet 105a, 105b, 105c, 105d is detected by an internal hall effect sensor 108a, 108b, 108c, 108d that measures gaussian force of a magnet 105a, 105b, 105c, 105d in the pivot arm to determine a distance the arm has moved. Those skilled in the art will recognize this movement also determines how much force has been applied to the arm based on how far the arm has moved. Thus, each sensor detects movement and force applied to its exterior. The combination of readings allows the device to calculate orientation and relative, three-dimensional movement at each sensor location. The cross-sectional view shown in FIG. 1D, illustrates the internal hall effect sensors 108b, 108d measure the Gaussian force of magnets 105b, 105dunder each leg for determining the distance upon which the leg has moved and how much force has been applied. These force readings can be used to assess bodily activation strength of the user for physical therapy and strength exercises.

Further, a printed circuit board (PCB) 107 is positioned under the housing 104 and is used for mounting the hall sensors 108a, 108b, 108c, and 108d, mounting a battery 109, a microprocessor 111 and other sensor electronics. A bottom lid 113 protects the PCB 107 by fastening around it and frictionally engaging with the housing 104. A charge port 115 enables the battery 109 to be easily charged for extended use.

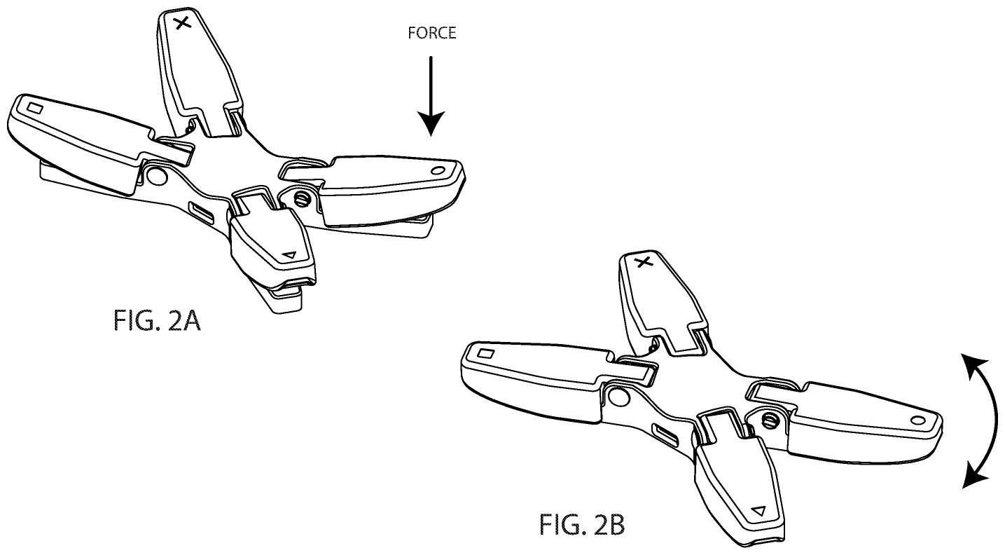

FIG. 2A and FIG. 2B are isometric views showing leg movement with forces applied where FIG. 2A shows the device in its default position while FIG. 2B shows the device in a fully engaged position. As seen in both FIG. 2A and 2B, each of the four legs has a spring-loaded pivot that holds it up at an angled position. When a force is applied to the top of a leg, the batten or leg rotates about its pivot point in a downward manner, closing the gap between the top and bottom of the assembly. The distance the leg travels is proportional to the amount of force applied e.g. for a lower force, the legs will move less; for a higher force, the legs may move all the way to the down position seen in FIG. 2B. When the force is removed, the spring returns the leg back to its original position. A hard stop prevents the leg from rotating further than its starting position. In order to calibrate the device, a calibration sequence measures an initial position, an activated position, and sometimes side-to-side movements to record and document specific movement and force measurements for that user's profile. The calibration sequence is used as a baseline for progress tracking and various exercises used in combination with the device.

Those skilled in the art will further recognize that magnets 105a, 105b, 105c, 105d each work to represent when and how much a user is engaging their muscles for a particular exercise. For example, movement is determined through muscle position, muscle force, movement and/or pressure against the device. The overall angle of the user when pressing, leaning, sitting, standing and/or laying down against a surface with any part of their body will provide data to each sensor for determining correct or incorrect position.

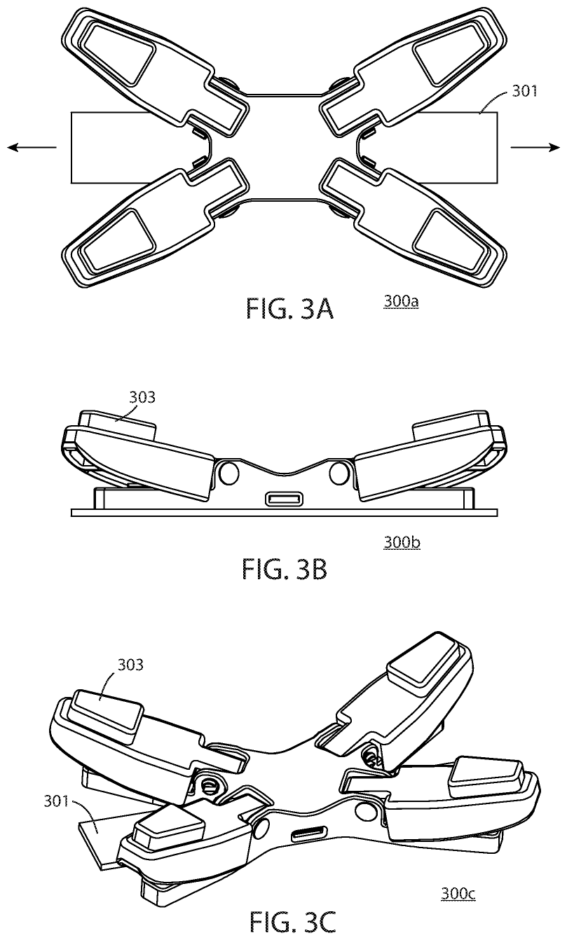

FIG. 3A, FIG. 3B and FIG. 3C are top, side and isometric views respectively of a muscle activation and movement detection device 300a, 300b, 300c according to a first alternative embodiment of the invention. In this embodiment, a strap 301 can be attached to the back of the device. The strap 301 allows the user to pre-place the device on their body before engaging in exercise. For example, this can be used on a person's lower back by first strapping the device to their back, in the proper location, and then sitting in a chair to complete an exercise. Further, optional adjustment pads 303 can be added to accommodate different applications and user sizes. The pads 303 come in multiple heights and can easily be added or removed.

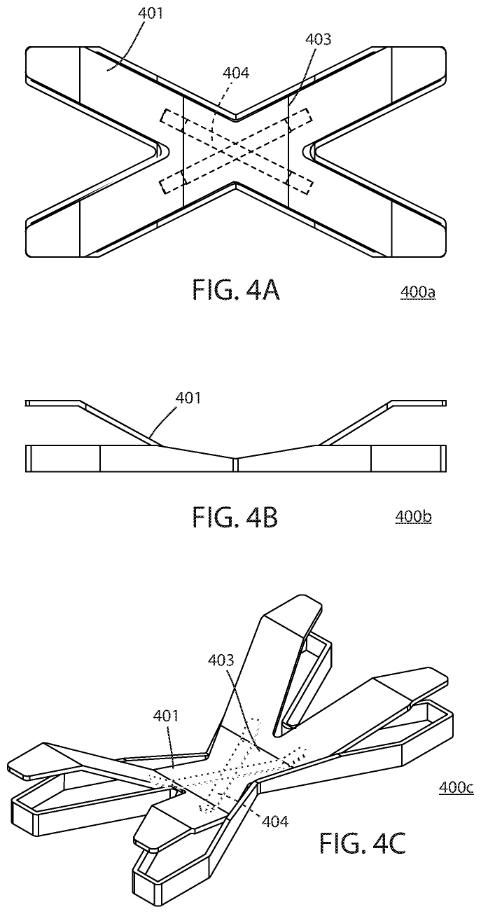

FIG. 4A, FIG. 4B are FIG. 4C are top, side and isometric view respectively of a muscle activation and movement detection device 400a, 400b, 400c according to a second alternative embodiment of the invention. In FIG. 4A, 4B and 4C, a different configuration of the main assembly is shown where the four pivot legs, as illustrated in the first embodiment, are replaced with a one-piece x-shaped plastic top housing 401. The top housing 401 flexes through a living hinge 403 to provide movement of the device. When a force is applied to the legs the hinge 403, the hinge 403 deflects in a downward direction. When this force is removed, the hinge naturally retracts, with each of the legs on the housing returning to their original position. To detect movement, either hall effect sensors with magnet or a flex-type sensor 404 may be used. Those skilled in the art will further recognize that a flex sensor measures how much the sensor has been flexed or bent and then outputs this information to a digital sensor where data can be read and decoded.

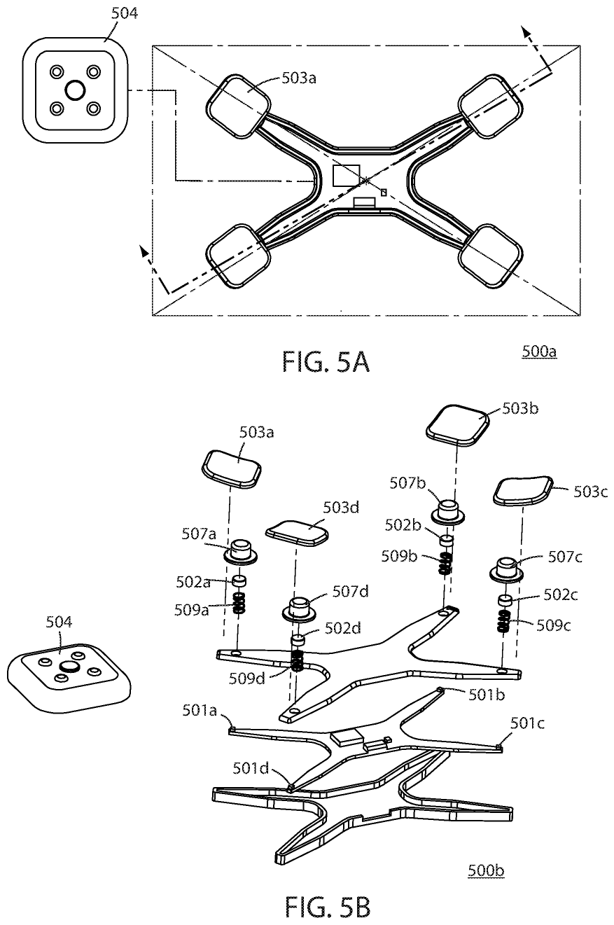

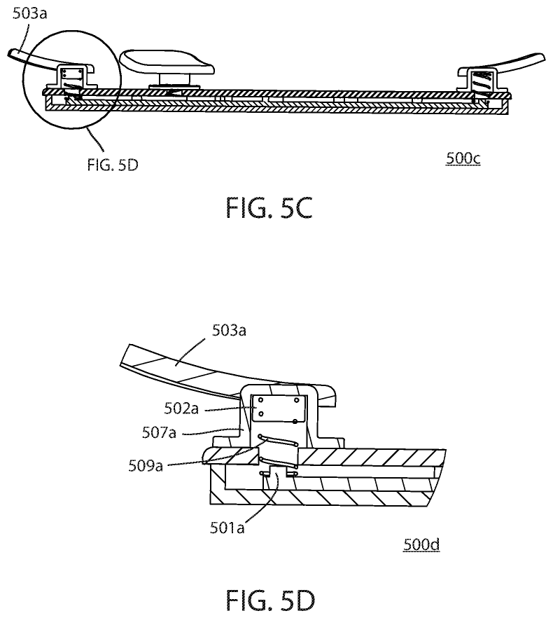

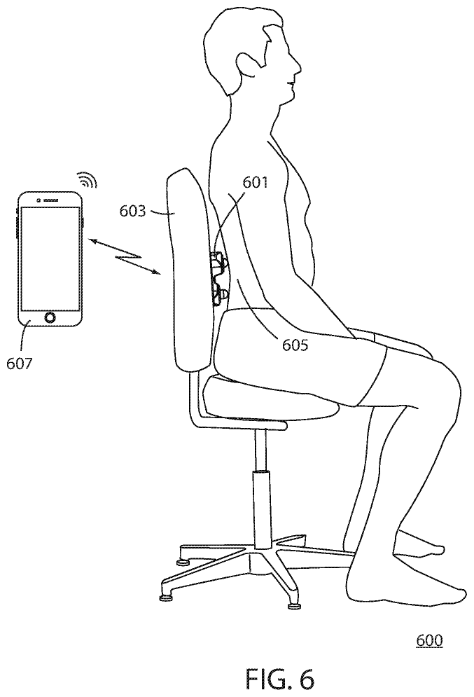

FIG. 5A, FIG. 5B, FIG. 5C are top, exploded and cross-sectional views respectively of a muscle activation and movement detection device according to a third alternative embodiment of the invention. FIG. 5D is a magnified view of the pad as seen in FIG. 5C. With regard to each of FIGS. 5A to 5D, these figures illustrate a low-profile feedback device 500a, 500b, 500c where the arms are removed and sensors 501a, 501b, 501c, and 501d detect movement at the four arms using magnets 502a. 502b, 502c, 502d. Those skilled in the art will further recognize that the device 500a, 500b, 500c can be enclosed in a cushion cover or alternatively can be embedded in an existing furniture product. For example, a sensors 501a, 501b, 501c, and 501d can detect either incremental movement or can be set to detect simple on/off detection. The initial calibration can be performed in software to select individual user measurements. Optional and customizable pads 503a, 503b, 503c, 503d are used to adapt the device to a certain user or setting. As seen in FIG. 5D, a plurality of caps 507a, 507b, 507c, 507dhold a respective magnet 502a, 502b, 502d, 502d in a fixed position under each pad 503a, 503b, 503c, 503d along with resilient members such as springs 509a, 509b, 509c, 509d.

In lieu of a customized software application or “App”, direct feedback from the device can also be achieved through either vibration, sound or light feedback. An ancillary mobile console 504 may also be connected as for easy visual feedback when used with a chair. For example, the device 500a, 500b, 500c can be placed on the seat back cushion and the mobile console 504 placed on the arm rest of the chair. A power switch allows the user to toggle the device on or off. Further, in other embodiments, the device can be placed on or in the seat of a chair or wheelchair. The device can monitor how long a user has been seated, as well as provide a basic four-zone pressure map of the user to ensure they are sitting in a balanced position.

FIG. 6 is a perspective view illustrating a muscle activation and movement detection, monitoring, tracking and alert device working in a chair system configuration 600. The device 601 works to detect and monitor muscle activation and movement for use in strength tracking, and alerting the user to various parameters. In use, the device 600 may be placed within a chair cushion or used as a stand-alone device against a chair back 603 while also in contact with the user's lower back 605. The device 601 includes a wireless transmitter enabling it to provide a wireless live feed of muscle forces, movement, angular positioning, exercises, and alerts, to the user's mobile device 607. This enables the user to strengthen their muscles through instantaneous feedback of user's body engagement and posture, tracking of stretching and strength exercises, and monitoring the progress of improved force profiles associated with muscle movement and activation for a particular physical exercise.

Thus, embodiments of the present invention are directed to a muscle activation and movement detection, monitoring, tracking and alert device and system that includes pivot arms formed into a cross-concave shape. Four sensors are configured underneath each end of the pivot arms for detecting positional forces against them. In use, the sensors measure force and movement to detect proper angular position, track exercise routines and durations, monitor muscular strength, and provide instant feedback through an alert to a user for improper position or to notify them they have completed an exercise routine, for example. It can also provide a wireless alert to the user when an improper angular position is detected. These allow the device to continually monitor and correct a user's position as well as track exercise routines and strength improvements, enabling them to effectively strengthen the specific area of their body they are working on in physical therapy or during personal at-home exercises.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

We claim:

1. A muscle activation and movement detection, monitoring, tracking and alert device comprising:
a plurality of semi-flexible pivot arms formed into a cross-shape;
a plurality of sensors for detecting forces under each of the plurality of pivot arms; and
wherein under each pivot arm includes at least one of the plurality of sensors configured substantially at its end to provide feedback to the user body positions and posture.

2. A core activation detection, monitoring, tracking and alert device as in claim 1, wherein the plurality of pivot arms is two.

3. A core activation detection, monitoring, tracking and alert device as in claim 1, wherein the plurality of sensors is four.

4. A core activation detection, monitoring, tracking and alert device as in claim 1, wherein the plurality of pivot arms are configured to be in a concave shape.

5. A core activation detection, monitoring, tracking and alert device as in claim 1, further comprising:
a wireless transmitter for providing an alert to a user's mobile device or computer.

6. A monitoring device for accessing a user physical position comprising:
a plurality of sensors;
a plurality of semi-flexible pivot arms above a respective sensor such that the plurality of pivot arms are configured into an X-shape that can adapt to a non-uniform surface for measuring the movements being applied at their surface.

7. A monitoring device as in claim 6, wherein the non-uniform surface is a person's back.

8. A monitoring device as in claim 6, wherein the non-uniform surface is a part of a person's body.

9. A monitoring device as in claim 7, where the device leverages the semi-flexibility of legs in the “X” shape to conform to the position of a person's back.

10. A monitoring device as in claim 6, where the device delivers consistent, accurate force readings that can be used to assess bodily activation strength of the user for physical therapy and physical strength exercises.

11. A monitoring device as in claim 6, wherein the device includes a calibration cycle that operates using both a user's unique anatomy and strength for developing a calibrated baseline that is used to develop specific strength exercises and performance tracking through a customized app.

12. A monitoring device as in claim 10, wherein the app is used by a user and physical therapists to encourage and track progress.

13. A core activation and detection device comprising:
a main body;
a plurality of flexible arms configured in an “X” shape on the main body; and
wherein the plurality of semi-flexible pivot arms are adapted to the contour of user's body in various settings and positions.

14. A core activation and detection device as in claim 13, wherein the device is manufactured in materials or geometry providing flexibility for user comfort and adaptability while remaining rigid for optimal sensor readings.

15. A core activation and detection device as in claim 13, wherein the design will amplify sensor readings using mechanical attributes of the pivot arm geometry.

16. A core activation and detection device as in claim 13, further comprising:
at least four sensors are configured on either side of the spine in distinct locations for offering an optimal measurement of the core activation exercise that is crucial for lower back pain physical therapy.

17. A core activation and detection device as in claim 13, further comprising:
a plurality of flexible sensors that move with the “X” shape on a user's back.

18. A core activation and detection device as in claim 13, wherein the device operates using a calibration sequence during device start-up for adapting sensor readings specifically for the user.

19. A muscle activation and detection device as in claim 18, wherein a calibration sequence measures an initial position, an activated position, and sometimes side-to-side movements to record and document specific movement and force measurements for that user's profile.

20. A muscle activation and detection device as in claim 18, wherein the calibration sequence is used as a baseline for progress tracking and exercises used with the device.
